SEQUENCE LISTING (3 sequences)

SEQ ID NO: 1 - protein, 8 aa, synthetic construct
  REGION 1..8: note = Synthetic peptide.
GGVLVQPG

SEQ ID NO: 2 - protein, 8 aa, synthetic construct
  REGION 1..8: note = Synthetic peptide.
  SITE 7: note = D-Proline
GGVLVQPG

SEQ ID NO: 3 - protein, 8 aa, synthetic construct
  REGION 1..8: note = Synthetic peptide.
  SITE 3: note = D-Valine
  SITE 4: note = D-Leucine
  SITE 5: note = D-Valine
  SITE 6: note = D-Glutamine
  SITE 7: note = D-Proline
GGVLVQPG

(19) United States
(12) Patent Application Publication
(10) Pub. No.: US 2025/0144166 A1
(43) Pub. Date: May 8, 2025

(54) COMPOSITIONS AND METHODS FOR INHIBITING RHO KINASE

(71) Applicants: INTERLUDE BIOPHARMA CO., Miami, FL (US); University of Maryland, College Park, College Park, MD (US)

(72) Inventors: Younggeon JIN, College Park, MD (US); Nir BARAK, Raleigh, NC (US); Jain KIM, College Park, MD (US)

(21) Appl. No.: 18/836,845

(22) PCT Filed: Feb. 9, 2023
(86) PCT No.: PCT/US2023/062247

§ 371 Date: Aug. 8, 2024

Related U.S. Application Data
(60) Provisional application No. 63/308,330, filed on Feb. 9, 2022.

(51) Int. Cl. A61K 38/08

(52) U.S. Cl. CPC A61K 38/08

(57) ABSTRACT

The present invention provides for compositions and methods for treating a condition characterized by Rho-associated coiled-coil kinase (ROCK) activity in a subject in need thereof via administration of an effective amount of larazotide or larazotide derivative, or pharmaceutically acceptable salt thereof, to said subject in an amount and manner effective to inhibit ROCK activity in a tissue.

### CROSS-REFERENCE TO RELATED APPLICATIONS

This application claims the benefit of U.S. Provisional Patent Application No. 63/308,330, filed Feb. 9, 2022, the entire contents of which are hereby incorporated by reference in their entirety.

### FIELD OF THE INVENTION

The present invention provides compositions, formulations, and methods for treating and preventing conditions associated with Rho-associated coiled-coil kinase (ROCK) activity.

### DESCRIPTION OF THE TEXT FILE SUBMITTED ELECTRONICALLY

This application contains a Sequence Listing in XML format submitted electronically herewith via Patent Center. The contents of the XML copy, created on Feb. 7, 2023, is named “NMT-038PC_116031-5038.xml” and is 6,919 bytes in size. The Sequence Listing is incorporated herein by reference in its entirety.

### BACKGROUND

Rho-associated coiled-coil containing kinases (ROCK) are effectors of the RhoA small GTPase, and have critical roles in facilitating actomyosin cytoskeleton contractility downstream of RhoA and RhoC activation. ROCK kinases are important for processes such as cell contraction, migration, apoptosis, survival, and proliferation. The two mammalian ROCK homologs, ROCK1 and ROCK2, are implicated in various disease processes such as cardiovascular disease, vascular injury, fibrosis, inflammatory bowel disease, and cancer, among others.

Pharmaceutical compositions and methods for inhibiting ROCK, for treatment or prevention of ROCK-related medical conditions, are desired.

### SUMMARY OF ASPECTS OF THE INVENTION

The present invention contemplates, in part, compositions and methods that are useful for the treatment of Rho-associated coiled-coil kinase (ROCK)-related conditions.

In various aspects, the invention provides a method for treating a subject for a condition characterized by Rho-associated coiled-coil kinase (ROCK) activity. The method comprises administering an effective amount of larazotide or larazotide derivative, or pharmaceutically acceptable salt thereof, to said subject in an amount and manner effective to inhibit ROCK activity in an organ or tissue. In various embodiments, the organ or tissue is selected from gastrointestinal tract, cancer tissue, eye, respiratory tract, and vasculature.

In some embodiments, larazotide or derivative thereof is administered to the gastrointestinal tract. In such embodiments, the subject may have a condition selected from cancer (e.g., GI cancer such as colorectal cancer or stomach cancer, or non-GI cancer), adenoma, inflammatory bowel disease (IBD) (e.g., Crohn's Disease or Ulcerative Colitis), celiac disease, esophagitis, inflammatory liver disease, kidney disease, pancreatitis, hyperglycemia, diabetes mellitus, and pulmonary or cardiac inflammation or fibrosis. In certain embodiments, the subject has or is at risk for colorectal cancer. For example, the subject's family history, genetic mutations, and/or health history may increase the subject's risk for colorectal cancer.

In other embodiments, larazotide or derivative is delivered directly to cancer tissue, which can be a primary tumor or metastatic tumor. For example, the subject may have a cancer selected from lung cancer, breast cancer, kidney cancer, liver cancer, prostate cancer, cervical cancer, colorectal cancer, pancreatic cancer, melanoma, ovarian cancer, bone cancer, urothelial cancer, gastric cancer, head and neck cancer, glioblastoma, head and neck squamous cell carcinoma (HNSCC), non-small cell lung carcinoma (NSCLC), small cell lung cancer (SCLC), bladder cancer, hormone-refractory prostate cancer, and lymphoma. In some embodiments, the tissue is metastatic cancer, such as metastatic melanoma. In some embodiments, the cancer is a sarcoma or carcinoma.

In other embodiments, larazotide or derivative is administered to the eye. For example, the subject may have an eye condition selected from Sjogren's syndrome, dry eye syndrome, age-related macular degeneration (AMD), macular edema, diabetic retinopathy, and glaucoma.

In some embodiments, larazotide or derivative is administered to the respiratory tract. For example, the subject may have a condition selected from asthma, chronic obstructive pulmonary disease (COPD), pulmonary fibrosis, cystic fibrosis, acute lung injury (ALI), acute respiratory distress syndrome (ARDS), emphysema, bronchitis, pneumonia, lung cancer, and respiratory infection.

In other embodiments, larazotide or derivative is administered to the vasculature or administered systemically. For example, the subject may have a condition selected from cardiac fibrosis, cardiac hypertrophy, hypertension, pulmonary hypertension, angina pectoris, vasospastic angina, heart failure, atherosclerosis, arteriosclerosis, diabetes mellitus, pancreatitis, kidney disease (including renal fibrosis), and stroke (for example, in prevention or stroke or stroke recovery).

In various embodiments, the larazotide derivative comprises one or more modifications that increase ROCK inhibitor activity as compared to larazotide. For example, in embodiments, the larazotide derivative comprises at least one, at least two, at least three, at least four, at least five (d)-amino acids. In certain embodiments, each amino acid of the larazotide derivative (other than Gly) is a (d)-amino acid, and the derivative is optionally a retro-inverso larazotide.

Other aspects and embodiments of the invention will be apparent from the following detailed description and working examples.

### BRIEF DESCRIPTION OF THE DRAWINGS

FIGS. 1A-B depict an evaluation of paracellular permeability during anoxia/reoxygenation (A/R) injury in larazotide acetate (LA) treated C2BBe1 intestinal cells. FIG. 1A depicts a schematic drawing showing the schedule of LA treatment and TEER measurement in A/R injury. FIG. 1B shows that treatment with 10 mM LA significantly (p<0.001) increases the TEER compared to non-treated A/R injured cells.

FIG. 2 shows that anoxia causes the myosin to be phosphorylated, resulting in internalization of the tight junction proteins occludin. The schematic shows how LA is predicted to prevent this phosphorylation.

FIG. 3 depicts the distribution of TJ proteins analyzed by immunoblotting from membrane and cytosol fractions and IF analyses after 1-hour reoxygenation. FIG. 3 shows the transmembrane protein occludin was significantly (p<0.05) internalized in anoxic injured cells compared to control cells. However, the occludin was significantly (p<0.05) increased in the membrane by 10 mM of LA compared to non-treated anoxic injured cells.

FIG. 4 shows the evaluation of the localization of tight junction and cytoskeleton proteins by immunofluorescence microscopic analyses at the 1-hour oxygenation. Permeable support membranes were fixed and stained for ZO-1 (red), occludin (green), and F-actin (purple). Immunolocalization of ZO-1 and occludin was analyzed by Z stack 3-D analysis. Whereas non-treated anoxic injured cells showed disruption of occludin, ZO-1, and F-actin, larazotide acetate (LA) treated cells revealed well-organized tight junction proteins and F-actin.

FIG. 5 depicts the ratio of pMLC-2/MLC-2 as evaluated by immunoblotting at the 1-hour reoxygenation time point. The expression of pMLC-2 was dramatically increased after A/R injury and significantly reduced by pre-treatment with 10 mM LA (##p<0.01).

FIG. 6 shows regulation of phosphorylation of MLC to regulate TJ barrier in intestinal epithelial cells. The TJ barrier is mainly determined by the phosphorylation level of the regulatory light chain of myosin (MLC) which is regulated by the two enzymes myosin light chain kinase (MLCK) and rho-associated coiled-coil protein kinase (ROCK). The increased intracellular Ca²⁺ levels stimulate MLCK activity. Enhanced Rho Kinase (ROCK) activity also directly phosphorylates MLC and inhibits myosin light chain phosphatase (MLCP) activity by phosphorylating the myosin phosphatase target subunit 1 (MYPT1).

FIG. 7 depicts inhibition of MLCK and ROCK increase TEER with or without larazotide compared to non-treated A/R injured cells. Pep 18 (MLCK inhibitor) treatment alone or combined with LA increased TEER. Fasudil (ROCK inhibitor)-treated monolayers showed increased TEER. Combined treatment of fasudil and LA increased TEER, however to a lesser extent than Fasudil treatment alone (*<p0.05, **p<0.01, ***p<0.005, ****p<0.0001).

FIG. 8 depicts possible mechanisms for regulation of phosphorylation of MLC to regulate paracellular actin myosin rings in intestinal epithelial cells. The TJ barrier is mainly determined by the phosphorylation level of the regulatory light chain of myosin (MLC), which is regulated by the two enzymes myosin light chain kinase (MLCK) and myosin light chain phosphatase (MLCP). These mechanisms can be regulated by various cellular pathways such as Ca²⁺ homeostasis, G-protein-coupled receptor (GPCR), and growth factor (GF) pathways.

FIGS. 9A-B shows different expression analyses using the next generation RNA-seq. FIG. 9A depicts a Venn diagram analysis of the quantity of the differentially expressed genes (DEGs) identified. FIG. 9B depicts hierarchical clustering of DEG, which was used to estimate expression patterns under different experimental conditions.

FIG. 10 depicts Gene Ontology (GO) functional classification analysis of (DEGs) from different combination of groups. It includes three main branches biological processes, cellular components, and molecular function. GO terms with padj<0.05 are significant enrichment. FIG. 10 shows 20 significantly enriched biological processes, cellular components and molecular functions.

FIG. 11 shows Kyoto Encyclopedia of Genes and Genomes (KEGG) pathway analysis of the enrichment of DEGs from different combination of groups. KEGG pathway terms with padj<0.05 are significant enrichment.

FIG. 12 shows the evaluation of paracellular permeability in C2BBe1 intestinal cells. Treatment with 10 mM LA significantly increased TEER compared to non-treated A/R injured cells at 1 hour after treatment.

FIG. 13 depicts the evaluation of proliferation of C2BBe1 intestinal cells. Treatment with 10 mM LA significantly increases proliferation compared to non-treated cells.

FIGS. 14A-B show the assessment of proliferation and migration C2BBe1 intestinal cells. FIG. 14A depicts that 10 mM LA-treated cells in regular media showed significantly increased wound healing at 48 hours. FIG. 14B shows that no significantly different migration patterns were detected between 10 mM LA-treated and non-treated cells in serum free media.

### DETAILED DESCRIPTION

The present invention provides compositions and methods for treating a condition characterized by Rho-associated coiled-coil kinase (ROCK) activity in a subject in need thereof via administration of an effective amount of larazotide or larazotide derivative, or pharmaceutically acceptable salt thereof, to said subject in an amount and manner effective to inhibit ROCK activity in a tissue.

The present disclosure shows that larazotide and larazotide derivatives, or pharmaceutically acceptable salts thereof, inhibit rho-associated protein kinases (ROCK). Inhibition of ROCK can provide therapeutic benefit for subjects having conditions associated with ROCK activity. ROCK and its downstream targets are involved in regulating actin cytoskeleton dynamics, and therefore are responsible for cell migration and motility. In addition, they are implicated in diverse biological processes such as cell junction integrity, cell cycle control, and cell apoptosis.

The ROCK enzyme has two isoforms: ROCK1 and ROCK2. Both kinases contain a catalytic kinase domain at the N terminus followed by a central coiled-coil domain, which includes the Rho-binding domain (RBD), and a C-terminal pleckstrin-homology (PH) domain.

The peptide agent known as larazotide has the amino acid sequence Gly-Gly-Val-Leu-Val-Gln-Pro-Gly (SEQ ID NO: 1). Larazotide promotes tight junction integrity of epithelial and endothelial tissues, including of the intestinal epithelium, and is being evaluated as a therapy for patients with celiac disease (CeD). In accordance with certain aspects and embodiments, the present invention provides larazotide derivatives that confer, among other things, increased resistance to exopeptidase degradation, including aminopeptidase degradation. In various embodiments, the larazotide derivative comprises one or more amino acid substitutions, deletions, and/or insertions with respect to the peptide having the amino acid sequence of SEQ ID NO: 1. Exemplary modifications are described in U.S. Pat. Nos. 8,785,374, 8,957,032, 9,279,807, PCT/US2019/19350, and PCT/US21/27410 (all of which are hereby incorporated by reference).

In some embodiments, the peptide derivative of larazotide contains one or more (d) amino acids. For example, the larazotide derivative may contain 1, 2, 3, 4, or 5 (d) amino acids (that is, the D configuration as opposed to the L configuration). In some embodiments, the larazotide derivative has the amino acid sequence of Gly-Gly-Val-Leu-Val-Gln-(d) Pro-Gly (SEQ ID NO: 2). This peptide is referred to herein as “(d)-Pro” or (d)-Pro larazotide. In other embodiments, the larazotide derivative has the amino acid sequence of Gly-Gly-(d) Val-(d) Leu-(d) Val-(d) Gln-(d) Pro-Gly (SEQ ID NO: 3). This peptide is referred to herein as “(d)-larazotide.” As demonstrated herein, (d)-larazotide is surprisingly effective at promoting tight junction integrity at substantially lower concentrations as well as higher concentrations, as compared to larazotide (which shows bell-shaped dose-response curve). This is a surprising observation, since typically, replacing L amino acids with D amino acids in peptide drugs will result in a loss of potency. That is, a peptide with D amino acids would be expected to bind with lower affinity to the receptor function, as compared to peptides having the natural L amino acids.

In various embodiments, the invention employs larazotide derivatives that are more effective at substantially lower doses or high doses as compared to larazotide, especially for administration to the gastrointestinal tract. Accordingly, the pharmaceutical compositions of the present invention can contain less than about 0.5 mg of the larazotide derivative. For example, in some embodiments, the pharmaceutical composition contains about 0.4 mg of the larazotide derivative or less, or about 0.3 mg of the larazotide derivative or less, or about 0.25 mg of the larazotide derivative of less, or about 0.2 mg of the larazotide derivative or less, or about 0.15 mg of the larazotide derivative or less, or about 0.1 mg of the larazotide derivative or less, or about 50 μg of the larazotide derivative of less, or about 25 μg of the larazotide derivative or less. In some embodiments, the pharmaceutical composition contains from about 50 μg to about 400 μg of the larazotide derivative or less, or from about 50 μg to about 200 μg of the larazotide derivative or less, or from about 50 μg to about 150 μg of the larazotide derivative or less.

In other embodiments, the present invention contemplates a pharmaceutical composition that contains more than about 0.5 mg of a larazotide derivative, and substantially avoids the inverse dose or “bell shape” response observed with larazotide. For example, in some embodiments, the pharmaceutical composition contains about 0.6 mg of the larazotide derivative or more, or about 0.75 mg of the larazotide derivative or more, or about 1.0 mg of the larazotide derivative or more, or about 1.25 mg of the larazotide derivative or more, or about 1.5 mg of the larazotide derivative or more, or about 2.0 mg of the larazotide derivative or more.

In some embodiments, the larazotide derivative (e.g., (d)-larazotide or (d)-Pro), is administered at about 0.5 mg. For example, the derivative can be more effective at 0.5 mg doses than larazotide.

The present invention provides methods and compositions for treating a condition associated with ROCK activity in a subject in need thereof by administering a composition comprising larazotide or larazotide derivative to a tissue having ROCK activity, thereby inhibiting ROCK activity in said tissue. The terms “subject” and “patient” are used interchangeably herein, and generally refer to mammalian subjects/patients. In various embodiments the subject is a human subject. Thus, the composition may be formulated and/or delivered by various routes to inhibit ROCK activity is a desired organ or tissue. In various embodiments, the pharmaceutical compositions comprising larazotide or a derivative thereof (or salt thereof) are administered, for example, to the gastrointestinal tract (GI) (e.g., enteral delivery), or by parenteral, intra-nasal, buccal, ophthalmic, or pulmonary delivery. Delivery can be local to affected tissues or systemic.

In some embodiments, the peptide or pharmaceutical composition is administered to the gastrointestinal tract (GI) of a subject.

In some embodiments, the subject has an inflammatory condition or injury to the gastrointestinal tract such as celiac disease, inflammatory bowel disease (IBD) (e.g., Crohn's Disease or Ulcerative Colitis), environmental enteropathy, esophagitis, necrotizing enterocolitis, and intestinal ischemia. In some embodiments, the subject has an adenoma (e.g., advanced adenoma), colorectal cancer, or stomach cancer. ROCK activity has been shown to be involved in tumor development. Wei L., et al., Novel Insights into the Roles of Rho Kinase in Cancer, Arch Immunol Ther Exp (Warsz). 2016; 64:259-278. In some embodiments, the subject has cancer, which can originate from any tissue, such as skin, colon, breast, lung, brain, bone, pancreas, kidney, liver, bladder, ovaries, testes, or prostate.

In other embodiments, the subject has leukemia, myeloma, or lymphoma.

In some embodiments, the subject is at risk for colorectal cancer. For example, in embodiments, the subject has an increased risk for colorectal cancer due to the subject's family history, genetic mutations, and/or health history (e.g., prior history of advanced adenoma or colorectal cancer). In some embodiments, the subject or a member of the subject's family has or had familial adenomatous polyposis (FAP), hereditary non-polyposis colorectal cancer (HNPCC), Peutz-Jeghers syndrome, or MUTYH-associated polyposis (MAP). In some embodiments, the subject has a mutation of one or more genes that increases risk for colorectal cancer, such as but not limited to APC, MLH1, MSH2, MSH6, PMS2, EPCAM, STK11 (LKB1), and MUTYH. For example, without being subject to limitation, the subject may have one or more single-nucleotide polymorphisms (SNPs) selected from rs6983267, rs4939827, rs3802842, rs16892766, rs10795668, rs4444235, rs10411210, rs6691170, rs4925386, rs3824999, rs647161, rs2423279, rs3217810, and rs59336. In some embodiments, the subject has undergone chronic antibiotic use. For example, chronic antibiotic use comprises use of an antibiotic regimen of at least 6 months, which can increase risk of CRC.

In some embodiments, the subject has a ROCK mutation that is associated with cancer development. Exemplary such mutations include Val1309, Tyr405, Ser1126, Pro1193S, relative to ROCK1 isoform, or Thr431Asn, Asp601Val, and Lys1083Met, relative to ROCK2 isoform.

In some embodiments, the subject is undergoing or has undergone a cancer treatment selected from one or more of chemotherapy, radiation, resection, and immunotherapy and immune-oncology agents. In embodiments, the cancer immunotherapy is therapy with an immune checkpoint inhibitor or immune stimulatory ligand, which include but are not limited to, inhibitors of: Programmed Death-Ligand 1 (PD-L1, also known as B7-Hl, CD274), Programmed Death 1 (PD-1), CTLA-4, PD-L2 (B7-DC, CD273), LAG3, TIM3, IDO1, IDO 2. T-cell stimulation ligands include, but are not limited to an agonist of CD28, OX-40, and ICOS. In certain embodiments, the subject receives an immune checkpoint inhibitor selected from an anti-CTLA-4, anti-PD-1, anti-PD-L1 and/or PD-L2 agent. In some embodiments, the immune checkpoint inhibitor is selected from ipilimumab, tremelimumab, pembrolizumab and nivolumab.

In still other embodiments, the subject has an inflammatory liver disease, kidney disease, pancreatitis, hyperglycemia, or pulmonary or cardiac inflammation or fibrosis. For example, in some embodiments, the subject has a fatty liver disease including, but not limited to NAFLD, NASH, alcoholic steatohepatitis (ASH), or a fatty liver disease resulting from hepatitis, obesity, diabetes, insulin resistance, hypertriglyceridemia, chronic kidney disease, IgA nephropathy (also known as Berger's disease), abetalipoproteinemia, glycogen storage disease, Weber-Christian disease, Wolmans disease, acute fatty liver of pregnancy, and lipodystrophy. See US 2019/0358289 and US 2021/0069286, which are hereby incorporated by reference in their entireties.

In some embodiments, the subject has a condition involving organ fibrosis, such as pulmonary fibrosis, cardio fibrosis, renal fibrosis, or hepatic fibrosis. Knipe R., et al. The Rho Kinases: Critical Mediators of Multiple Profibrotic Processes and Rational Targets for New Therapies for Pulmonary Fibrosis, Pharmacol Rev. 2015 January; 67 (1): 103-117. In some embodiments, the subject has a condition associated with pulmonary fibrosis, such as chronic obstructive pulmonary disease (COPD), cystic fibrosis, acute lung injury (ALI), acute respiratory distress syndrome (ARDS), emphysema, bronchitis, asthma, pneumonia, and respiratory infection.

In other embodiments, the larazotide or derivative is administered directly to cancer tissues, including but not limited to intratumoral administration or via encapsulation or conjugation to nanoparticles, or other means. ROCK enzymes function in various processes of cancer progression, such as tumor invasion and metastasis, proliferation, and apoptosis or survival, and impact both cancer and cancer-associated cells, such as fibroblasts and endothelial cells.

In embodiments, the tissue is primary cancer. A primary cancer refers to cancer cells at an originating site that become clinically detectable, and may be a primary tumor. For example, the cancer may be Stage I or Stage II cancer. In some embodiments, the tissue is primary cancer and the cancer is selected from lung cancer, breast cancer, kidney cancer, liver cancer, prostate cancer, cervical cancer, colorectal cancer, pancreatic cancer, melanoma, ovarian cancer, bone cancer, urothelial cancer, gastric cancer, head and neck cancer, glioblastoma, head and neck squamous cell carcinoma (HNSCC), non-small cell lung carcinoma (NSCLC), small cell lung cancer (SCLC), bladder cancer, hormone-refractory prostate cancer, and lymphoma.

In embodiments, the tissue is metastatic cancer. “Metastasis” refers to the spread of cancer from a primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. In some embodiments, the tissue is metastatic cancer, such as metastatic melanoma. In various embodiments, the patient has a cancer that is sarcoma or carcinoma.

In some embodiments, the subject is undergoing or has undergone a cancer treatment selected from one or more of chemotherapy, radiation, resection, and immunotherapy and immune-oncology agents. In embodiments, the cancer immunotherapy is therapy with an immune checkpoint inhibitor or immune stimulatory ligand, which include but are not limited to, inhibitors of: Programmed Death-Ligand 1 (PD-L1, also known as B7-Hl, CD274), Programmed Death 1 (PD-1), CTLA-4, PD-L2 (B7-DC, CD273), LAG3, TIM3, IDO1, IDO 2. T-cell stimulation ligands include, but are not limited to agonist of CD28, OX-40, and ICOS. In certain embodiments, the subject receives an immune checkpoint inhibitor selected from an anti-CTLA-4, anti-PD-1, anti-PD-L1 and/or PD-L2 agent. In some embodiments, the immune checkpoint inhibitor is selected from ipilimumab, tremelimumab, pembrolizumab and nivolumab.

In some embodiments, the larazotide or derivative is administered to the eye, such as the surface of the eye, or by intravitreal injection.

In embodiments, the subject has Sjogren's syndrome (SS). Sjogren's syndrome is an autoimmune condition in which the moisture-producing glands of the body are affected, resulting in the development of, for example, dry mouth (xerostomia), dry eye syndrome (e.g., chronic dry eye and/or xerosis), and/or dry skin (e.g., xerosis), as well as other symptoms. In accordance with embodiments of the invention, the Sjogren's syndrome can be primary Sjogren's syndrome, or may be secondary Sjogren's syndrome, that is, occurring in association with another autoimmune or connective tissue disorder. The inflammation resulting from SS progressively damages glands, and is characterized by lymphocyte infiltration within the glands, elevated levels of B-cell activating factor (BAFF), as well as autoantibody production (e.g., anti-SSA/Ro). See, Nair J J and Singh T P, Sjogren's syndrome: Review of the etiology pathophysiology & potential therapeutic interventions. J. Clin. Exp. Dent. 2017; 9 (4): e584-9. In exemplary embodiments, the larazotide or derivative is administered to the surface of the eye, for the treatment of primary or secondary Sjogren's syndrome.

In some embodiments, the subject has glaucoma. ROCK inhibitors are a promising treatment option for lowering intraocular pressure (IOP) in glaucoma, for example. For example, in some embodiments, glaucoma is treated by administering larazotide or derivative thereof (or salt thereof) to the ocular surface.

In other embodiments, the subject has an inflammatory eye disease such as macular degeneration, macular edema, or diabetic retinopathy. Age-related macular degeneration (AMD) is a blinding eye disease which gradually increases with age. Inflammation participates in AMD pathogenesis, including choroidal neovascularization and geographic atrophy. In some embodiments, larazotide or derivative thereof is administered by intraocular administration (e.g., intravitreal injection) to the back of the eye.

In some embodiments of the invention, the larazotide or derivative is administered to the respiratory tract. See U.S. Pat. No. 10,723,763; U.S. Provisional Application No. 63/181,486; and PCT/US21/2741, all of which are hereby incorporated by reference. In embodiments, the subject has a condition selected from chronic obstructive pulmonary disease (COPD), pulmonary fibrosis, cystic fibrosis, acute lung injury (ALI), acute respiratory distress syndrome (ARDS), emphysema, bronchitis, asthma, pneumonia, lung cancer, and respiratory infection.

In embodiments, the larazotide or larazotide derivative is formulated for administration as a solution aerosol or powder to the lungs.

In other embodiments, the larazotide or larazotide derivative is administered to the nasal epithelium as a nasal solution or nasal emulsion.

In some embodiments, the larazotide or derivative is administered to the vasculature. For example, the larazotide or derivative can be administered systemically, or locally applied by a catheter. For example, the subject may have a condition selected from cardiac fibrosis, cardiac hypertrophy, hypertension, pulmonary hypertension, angina pectoris, vasospastic angina, heart failure, atherosclerosis, arteriosclerosis, diabetes mellitus, pancreatitis, kidney disease (including renal fibrosis), and stroke (for example, in prevention or ischemic stroke or stroke recovery).

Pharmaceutical compositions provided herein can be formulated for release in affected portions of the GI (e.g., stomach, small intestine, and/or large intestine). In other embodiments, larazotide or derivatives are administered systemically (e.g., intravenously or by subcutaneous injection). In some embodiments, the peptide composition is administered to the lungs as a solution aerosol or powder. In some embodiments, the peptide composition is administered to the nasal epithelium as a nasal solution or nasal emulsion. In some embodiments, the peptide composition is administered to the oral cavity or esophagus as a liquid or orally disintegrating tablet. In some embodiments, the peptide composition is administered to the ocular surface or intraocularly.

Larazotide derivatives of the present invention may be administered in any suitable form, including as a salt. For example, peptides may be administered as an acetate salt. Alternative salts may be employed, including any pharmaceutically acceptable salt such as those listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In various embodiments, the peptides are formulated as pharmaceutical compositions, which can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, modified 1 release formulations, controlled-release formulations, or any other form suitable for use.

In some embodiments, the pharmaceutical compositions are formulated as a composition adapted for parenteral administration. Dosage forms suitable for parenteral administration (e.g. intravenous, intramuscular, subcutaneous or intraperitoneal injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents. In these embodiments, the compositions can be effective for treating conditions involving systemic inflammation or injured or inflamed endothelial tissue.

In some embodiments, the compositions are administered to a subject by contacting the epithelial tissues or mucosal surfaces of the gastrointestinal tract. For example, the compositions may be formulated for delivery to one or more of the stomach, small intestine, and/and large intestine. By targeting release of the peptide in the affected region(s) (e.g. duodenum, jejunum and ileum, colon transversum, colon descendens, colon ascendens, colon sigmoidenum and cecum), ROCK inhibition at any location in the GI can be effected. Targeted delivery of the peptide in the small or large intestine can be achieved by coating beads or particles with the peptide, along with a delayed-release coating that prevents release in the stomach and degrades at or near the targeted location(s).

In some embodiments, the larazotide or larazotide derivative is administered in a sustained release or controlled release formulation that releases from about 0.5 to about 5 mg of larazotide or derivative in the intestine. In certain embodiments, the controlled release formulation contains at least 0.5 or 1 mg of larazotide or derivative.

In some embodiments, the peptide (e.g., larazotide or larazotide derivative) is formulated for sustained or modified or controlled delivery in one or more locations of the GI. For example, the present invention contemplates a sustained or controlled release formulation that may functionally release the peptide in the small and/or large intestine over the course of at least about 2 hours, or over the course of at least about 2.5 hours, or over the course of at least about 3 hours, or over the course of at least about 4 hours, or over the course of at least about 5 hours. In some embodiments, the sustained or controlled release composition begins to release peptide starting within about 10 to about 30 minutes of exposure to simulated intestinal fluid, with release of peptide continuing for at least about 180 minutes, or at least about 210 minutes, or at least about 240 minutes, or at least about 280 minutes of exposure to simulated intestinal fluid. Release profiles can be prepared, for example, using compositions with different enteric polymer coats and/or different thicknesses of the polymer coats. In some embodiments, the invention provides a composition comprising an effective amount of larazotide or derivative (or salt thereof), contained within a biodegradable or erodible polymer matrix, which further comprises an enteric coating. Formulations employing a biodegradable or erodible matrix are described in WO 2021/034629, which is hereby incorporated by reference in its entirety. Further, the erodible polymer matrix can comprise a polysaccharide matrix. In some embodiments, the matrix comprises one or more of cellulose, chitin, chitosan, alginate, amylose, pectin, callose, laminarin, chrysolaminarin, xylan, arabinoxylan, mannan, fucoidan, galactomannan, xanthan gum, dextran, welan gum, gellan gum, diutan gum, pullulan, hyaluronic acid, and derivatives thereof. In further embodiments, the matrix comprises microcrystalline cellulose. In these embodiments, the composition leverages the low effective dose of larazotide or a larazotide derivative (e.g., (d)-larazotide or (d)-Pro), while also minimizing any local accumulation of inactive fragments. Further, the formulation in these embodiments has the benefit of treating large surfaces of the GI with small doses of the peptide deposited continually during transit.

In various embodiments, the pharmaceutical composition may be formulated to have a delayed-release profile, i.e. not immediately release the active ingredient(s) upon ingestion; rather, postponement of the release of the active ingredient(s) until the peptide passes the stomach and is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon). In an embodiment, the pharmaceutical composition is formulated to have a delayed-release profile as described in, for example, U.S. Pat. No. 8,168,594, the entire contents of which are hereby incorporated by reference.

For example, the peptide may be administered to at least the duodenum of the patient, as an oral dosage, delayed-release composition that contains the peptide. In such embodiments, the composition comprises a first population of beads having a coating that is stable in gastric fluid and unstable in intestinal fluid so as to degrade and substantially release the peptide in the duodenum. The composition may further comprise a second population of beads with a pH-dependent coating to affect release of the peptide in the jejunum and/or ileum of the patient. For example, the second population of beads may release the peptide about 30 minutes or about 45 minutes after the beads releasing peptide in the duodenum. The oral dosage composition can be in the form of a capsule or tablet. The pH-dependent coating in some embodiments is a 1:1 co-polymer of methacrylic acid and ethyl acrylate, wherein the thickness of the layer determines the release profile of each bead. The beads may have one or more additional coatings such as a base coat, a separating layer, and an overcoat layer. In these embodiments, the contents of the beads will be released in a more bolus manner at targeted locations, but the properties of (d)-larazotide or (d)-Pro will be more effective than larazotide with such release profiles.

In an exemplary oral dosage composition, an effective amount of the peptide (e.g., as the acetate salt) is provided in first delayed-release particles that are capable of releasing the peptide in the duodenum of a patient, and second delayed release particles that are capable of releasing the peptide in the jejunum of a patient. Each particle has a core particle, a coat comprising the peptide (e.g., (d)-larazotide or (d)-Pro) over the core particle, and a delayed-release coating (e.g., a 1:1 co-polymer of acrylate and methacrylate) outside the coat comprising the peptide. Whereas the first delayed-release particles release at least 70% of the peptide in the first delayed-release particles by about 60 minutes of exposure to simulated intestinal fluid having a pH of greater than 5; the second delayed-release particles release at least 70% of the peptide by about 30 and about 90 minutes of exposure to simulated intestinal fluid having a pH of greater than 5.

Generally, the delayed-release coating may degrade as a function of time without regard to the pH and/or presence of enzymes. Such a coating may comprise, for example, a water insoluble polymer. Its solubility is therefore independent of the pH. The term “pH independent” as used herein means that the permeability of the polymer and its ability to release pharmaceutical ingredients is not a function of pH and/or is only very slightly dependent on pH. Such coatings may be used to prepare, for example, sustained release formulations. Suitable water insoluble polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like.

Other examples of polymers for constructing delayed release coatings include, but are not limited to, lacquer, and acrylic and/or methacrylic ester polymers, polymers or copolymers of acrylate or methacrylate having a low quaternary ammonium content, or mixture thereof and the like. Examples of insoluble polymers include EUDRAGIT RS®, EUDRAGIT RL®, and EUDRAGIT NE®. Insoluble polymers include, for example, polyvinyl esters, polyvinyl acetals, polyacrylic acid esters, butadiene styrene copolymers, and the like.

Various types of enteric coatings for delayed yet substantial delivery of active agents to the GI tract are known. In some embodiments, the sustained-release composition includes an enteric agent that is substantially stable in acidic environments and substantially unstable in near neutral to alkaline environments. In an embodiment, the sustained-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymer include, for example, EUDRAGIT® FS 30D, L 30 D-55, L 100-55, L 100, L 12.5, L 12.5 P, RL 30 D, RL PO, RL 100, RL 12.5, RS 30 D, RS PO, RS 100, RS 12.5, NE 30 D, NE 40 D, NM 30 D, S 100, S 12.5, and S 12.5 P. In some embodiments, one or more of EUDRAGIT® FS 30D, L 30 D-55, L 100-55, L 100, L 12.5, L 12.5 P RL 30 D, RL PO, RL 100, RL 12.5, RS 30 D, RS PO, RS 100, RS 12.5, NE 30 D, NE 40 D, NM 30 D, S 100, S 12.5 and S 12.5 P is used. The enteric agent may be a combination of the foregoing solutions or dispersions. In some embodiments, the enteric agent is EUDRAGIT F30D, which comprises a co-polymer of methyl acrylate, methyl methacrylate, and methacrylic acid. The co-polymer has a ratio of free carbonyl groups to ester groups of about 1:10.

In some embodiments, the beads comprise an enteric coating that is substantially resistant to dissolution in simulated gastric fluid. The composition remains essentially intact, or may be essentially insoluble, in gastric fluid. The stability of a gastric-resistant coating can be pH dependent. For example, the enteric coating may prevent substantial release of the peptide in simulated gastric fluid as well as simulated intestinal fluid having a pH of about 5.5. In some embodiments, the matrix provides for the sustained release of the peptide in simulated intestinal fluid having a pH of about 6 or more, such as from about 6.5 to about 7.0. Thus, the enteric coating is stable in simulated gastric fluid but unstable in simulated intestinal fluid having a pH above about 6.0. The enteric coating in such embodiments does not substantially release peptide in the duodenum, but delays release until the composition enters the jejunum, and thereafter providing a sustained release in the jejunum and ileum.

In some embodiments, the composition is a capsule for oral delivery comprising a population of beads, the population of beads comprising an effective amount of the larazotide or derivative (e.g., (d)-larazotide or (d)-Pro or salt thereof) contained within an erodible polymer matrix, the beads further comprising an enteric coating, which may comprise a co-polymer of methyl acrylate, methyl methacrylate, and methacrylic acid. The ratio of free carbonyl groups to ester groups in the co-polymer may be about 1:10 (e.g., EUDRAGIT F30D). In such embodiments, the enteric coating may be from about 20% to about 30% of the total weight of the composition. In some embodiments, the erodible matrix comprises microcrystalline cellulose. In some embodiments, the composition provides for less than about 15% release of peptide after about 2 hours in simulated gastric fluid. Further, the composition provides for less than about 25% release of peptide after about 2 hours in simulated intestinal fluid having a pH of about 5.5. In various embodiments, the composition releases at least about 40% but no more than about 80% of peptide after about 2 hours in simulated intestinal fluid having a pH of about 7.0. In various embodiments, 100% release in simulated intestinal fluid having a pH of about 7 is not reached until at least three hours, or in some embodiments, at least about 3.5 or at least about four hours.

In some embodiments, the pharmaceutical composition involves a coated tablet, or coated beads or granules, having a delayed-release profile as described in, for example, U.S. Pat. No. 8,168,594, the entire contents of which are hereby incorporated by reference. An exemplary enteric coating comprises a co-polymer of acrylate and methacrylate, which is a 1:1 co-polymer in some embodiments. Other fillers, binder, and plasticizers, including for seal coats or top coats, are described in U.S. Pat. No. 8,168,594, which is hereby incorporated by reference.

In accordance with certain embodiments, the present invention provides for the composition described herein to be administered one or more times daily. For example, the composition may be administered about once daily, about two times daily, or about three times daily. In various embodiments, a once daily to three times daily regimen is continued for a prolonged period. In some embodiments, the composition is administered every day. In other embodiments, the larazotide or larazotide derivative composition is administered 1 to 3 times weekly. In some embodiments, the regimen is continued for at least about 1 month, at least about 2 months, at least about 4 months, at least about 6 months, or at least about 8 months. In some embodiments, treatment is continuous to delay or prevent disease progression or to reduce or ameliorate symptoms of a chronic disease.

### EXAMPLES

### Example 1: Elucidation of the Mechanism of Action of Larazotide Acetate in Intestinal Barrier Function

This Example provides the results of an investigation of the role of larazotide acetate in the regulation of phosphorylation of MLC-2 during A/R injury. The results indicate that apically treated larazotide acetate on A/R injured Caco-2BBe1 monolayer protected epithelial barrier functions via regulating TJ proteins and actin stabilization. In addition, the larazotide acetate treatment also reduced increased phosphorylation of MLC-2 during A/R injury to regulate the epithelial barrier functions.

### A. Evaluation of Barrier Function

Pre-treatment of human intestinal epithelial cells with larazotide acetate was shown to tighten the intestinal barrier during anoxia/reoxygenation (A/R) injury. The perijunctional actomyosin ring contracts in response to phosphorylation of myosin light chain-2 (MLC-2) resulting the internalization of the TJ transmembrane proteins, and therefore this study evaluates whether larazotide acetate would protect the TJ barrier during anoxia/reoxygenation (A/R) injury via inhibition of MLC-2 phosphorylation.

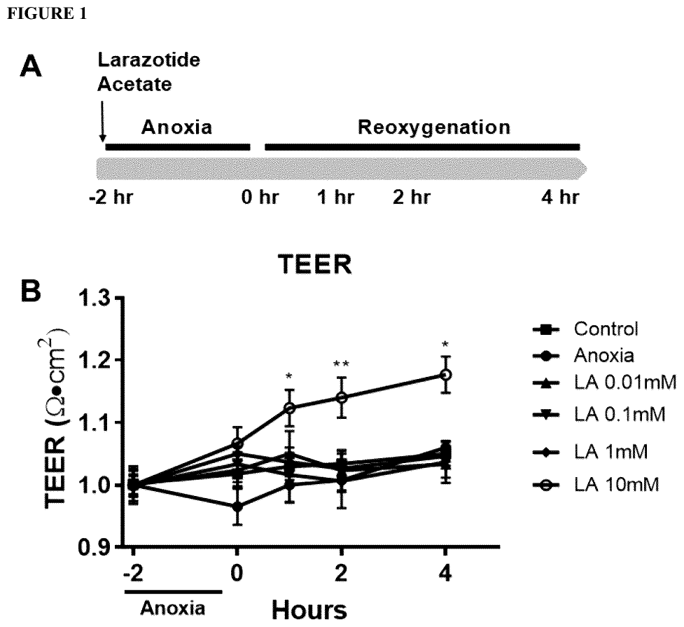

Specifically, to evaluate barrier function, transepithelial electrical resistance (TEER) was measured in anoxia/reoxygenation (A/R)-injured C2BBe1 (Caco-2 brush border-expressing Caco-2) cells treated with or without larazotide acetate (LA). Monolayers of C2BBe1 were treated with LA (0.001, 0.1, 1, and 10 mM) and were subjected to anoxia for 2 hours followed by reoxygenation with 21% O₂. See FIG. 1A. To generate A/R injury, C2BBe1 cells were placed in a modular incubator chamber (Billups-Rothenberg, San Diego, CA) and flushed with 95% N2/5% CO₂ for 5 minutes. The modular chamber was then sealed airtight and placed in an incubator for 2 hours. After 2 hours, the cells were removed from the modular incubator chamber and placed in a 21% O₂ normal environment. TEER was measured to assess barrier function during A/R injury and recovery. The TEER was measured using a Chopstick Electrode Set (WPI, LLC, Sarasota, FL) on the basal and apical sides of a monolayer and attached to an Epithelial Volt Ohm Meter2 (WPI, LLC, Sarasota, FL).

The resulting TEER was shown to be significantly increased (p<0.001) in C2BBe1 monolayers treated with 10 mM of LA compared to control cells. FIG. 1B. The increasing TEER is evidence of the ability of LA to close the “leaky” tight junction barrier induced by anoxic injury.

### B. Evaluation of Tight Junctions and MLC Activity

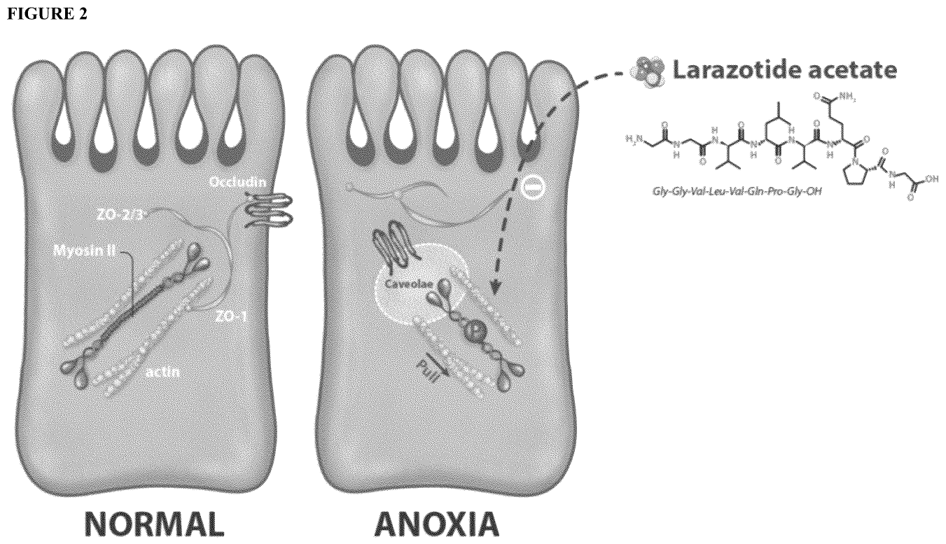

As depicted in FIG. 2, A/R injury induces phosphorylation of myosin light chain-2 (MLC-2) to internalize the tight junction proteins. The aim of this experiment was to determine whether LA aids inhibition of phosphorylated MLC-2 to protect tight junction barrier during A/R injury. To this end, localization tight junction proteins, structure of actin, and pMLC-2 were studied.

Specifically, the distribution of tight junction proteins and actin structure was evaluated using western blots on membrane and cytosol fractions and immunofluorescence microscope analysis. Mem-PER Eukaryotic Membrane Protein Extraction Reagent Kit (Thermo Scientific) was used to fractionate membrane and cytosolic compartment. The extracted proteins were blotted with primary antibodies anti-ZO-1 and anti-occludin (Invitrogen). The cold methanol fixed monolayers were stained with primary antibodies (ZO-1 and occludin) and phalloidin (F-actin) for immunofluorescence microscope analysis. The stained monolayers were examined with an Olympus IX83 Inverted Motorized Microscope with cellSens software (Olympus, Tokyo, Japan). pMLC-2/MLC-2 expression was evaluated using western blot analysis with primary antibodies for pMLC-2 and MLC-2 (CST, Danvers, MA).

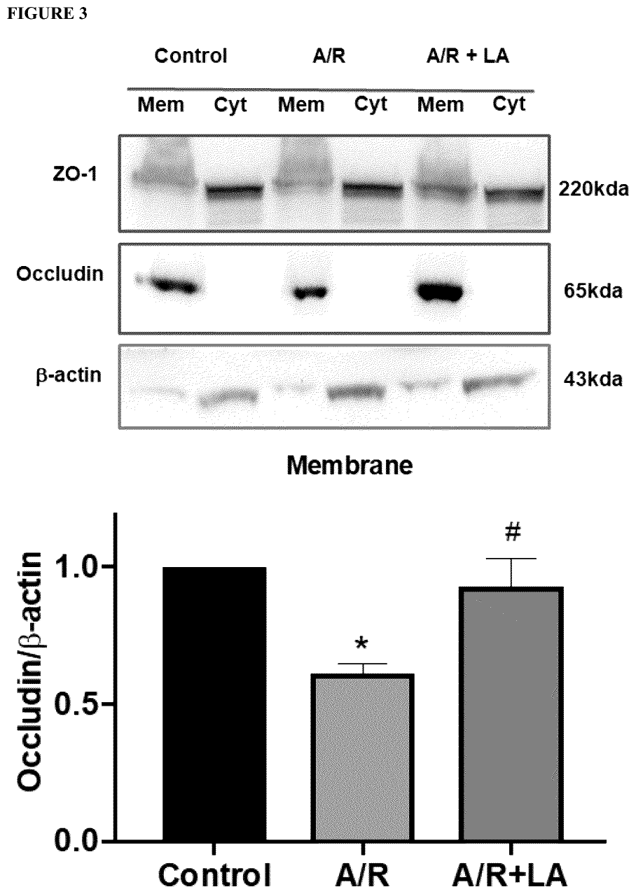

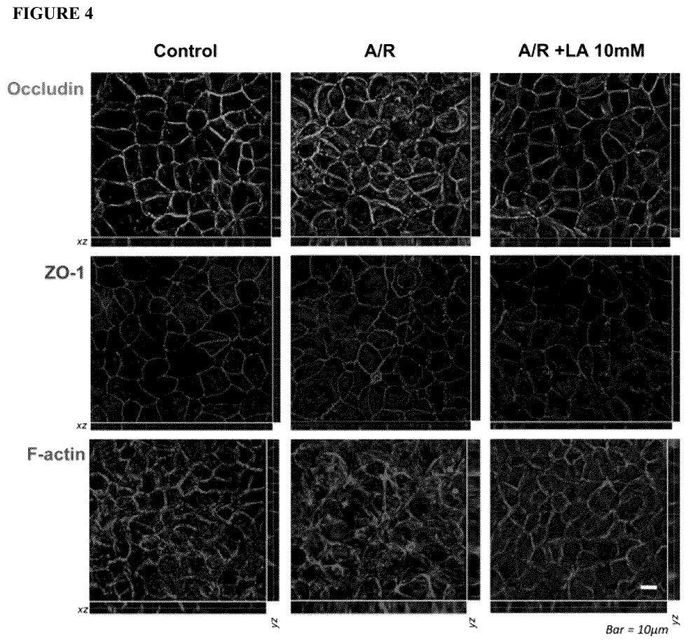

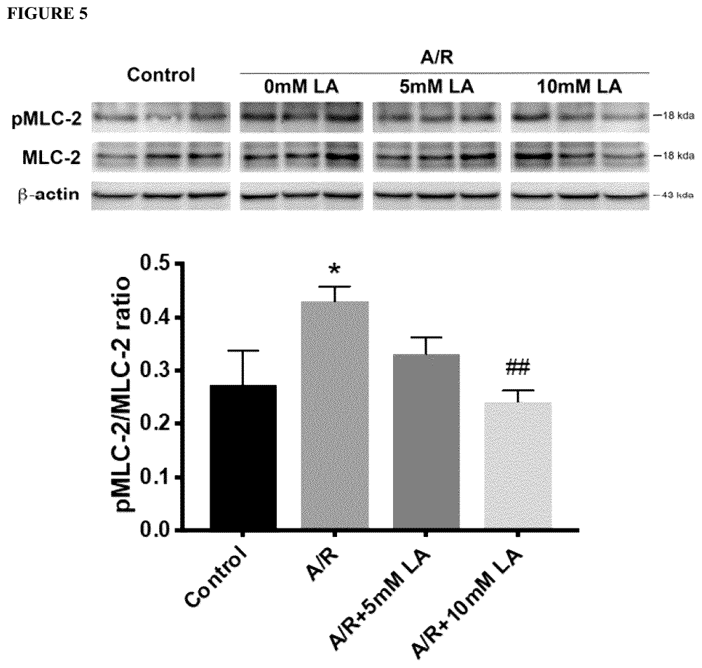

The data showed that tight junction protein occludin was internalized into the cytosol and ZO-1 was disrupted during A/R injury. However, as depicted in FIG. 3, treatment with 10 mM LA prevented disruption of tight junction proteins during A/R injury. F-actin structure was also disrupted during A/R injury and protected with 10 mM LA. See FIG. 4. In addition, FIG. 5 shows that pMLC-2 was significantly increased by A/R injury and treatment with 10 mM LA significantly reduced this phosphorylation. Taken together, the data suggest that LA protects the tight junction barrier during A/R injury by decreased pMLC-2.

Treatment of LA increased tight junction barrier in human intestinal cells during A/R injury. It was shown that tight junction proteins were protected by treatment with LA in C2BBe1 cells during A/R injury. The anoxia-induced increased phosphorylated MLC-2 during A/R injury and phosphorylated MLC-2 was significantly reduced by LA.

Distribution of tight junction proteins were protected by treatment with LA in human intestinal epithelial cells during A/R injury. This enhanced barrier function during A/R injury induced by LA treatment was caused by reduced phosphorylated MLC-2 with actin stabilization.

### C. Evaluation of MLCK and ROCK Activity

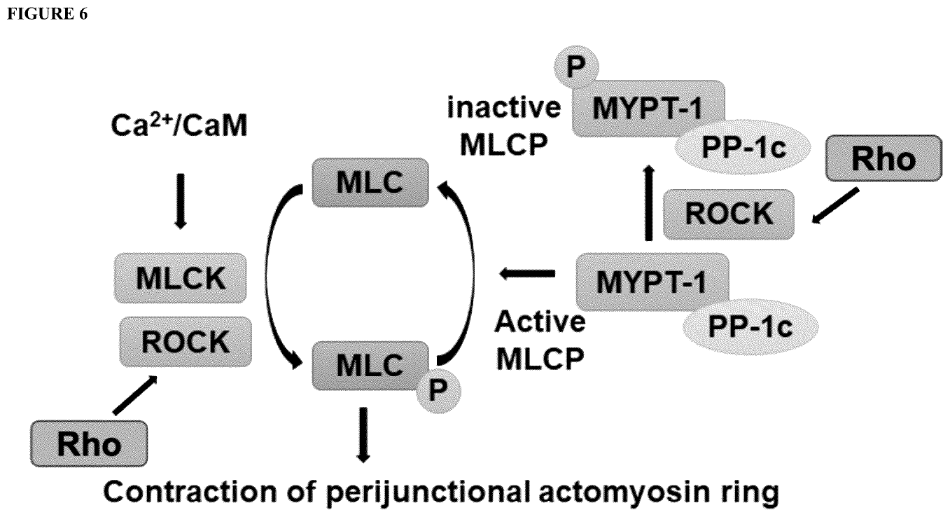

FIG. 6 shows that phosphorylated myosin light chain (pMLC) is regulated by myosin light chain kinase (MLCK), myosin light chain phosphatase (MLCP), and Rho kinase (ROCK). The purpose of this example was to elucidate the role of LA in kinase activity by performing kinase inhibition experiments.

To evaluate barrier function, the TEER was measured in A/R-injured C2BBe1 cells treated with or without LA. Monolayers of C2BBe1 were treated with 10 mM LA, 1 μM Y-27632 dihydrochloride (ROCK inhibitor, ApexBio, Houston, TX), and 400 μM Peptide 18 (MLCK inhibitor, Tocris Bioscience, Minneapolis, MN), and then were subjected to anoxia for 2 hours followed by reoxygenation with 21% O₂.

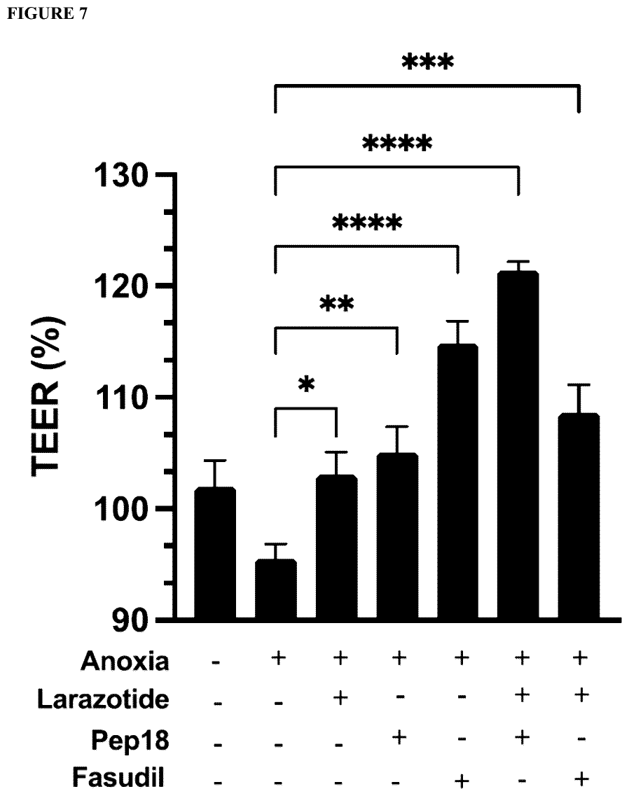

As shown in FIG. 7, Peptide 18 treatment either alone or with LA in A/R injured cells increased TEER values compared to non-treated A/R injured cells. In particular, combination treatment with Peptide 18 and LA showed a synergistic effect in A/R injured cells. However, Y-27632 treated in conjunction with LA did not show any additive or synergistic effects.

Based on the substantial increase in TEER when LA and Peptide 18 were applied together in A/R injured cells, it is presumed that LA acts on a separate pathway then MLCK. TEER levels in LA-treated A/R injured cells and LA and Y-27632-treated A/R injured cells were similar. These data indicate that the reduced phosphorylation of MLC-2 by LA may be regulated through the ROCK activity.

### D. RNAseq Analyses

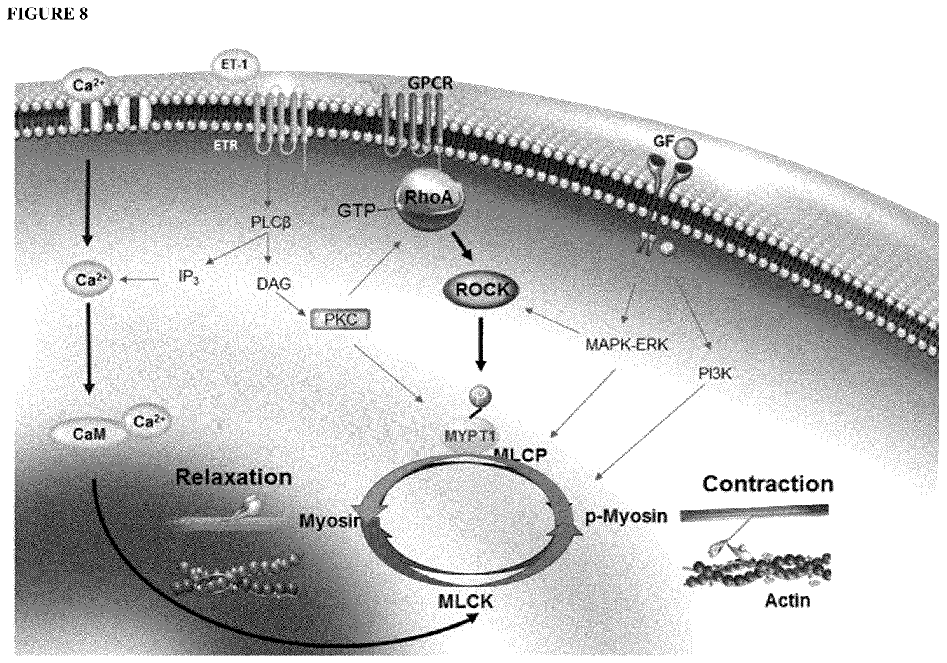

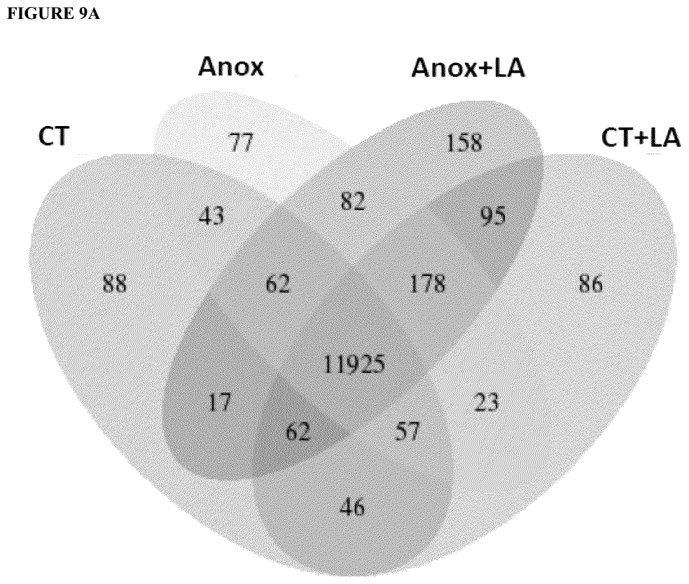

Since the phosphorylation of MLC can be regulated by various cellular pathways, as shown by FIG. 8, high-throughput RNAseq analysis was performed to elucidate the mechanism of action of LA. Specifically, monolayers of C2BBe1 were treated with 10 mM LA and were subjected to anoxia for 2 hours followed by reoxygenation with 21% O₂ for 1 hour. Next-generation RNA sequencing and biostatistical analyses were employed to assess cellular regulatory pathways.

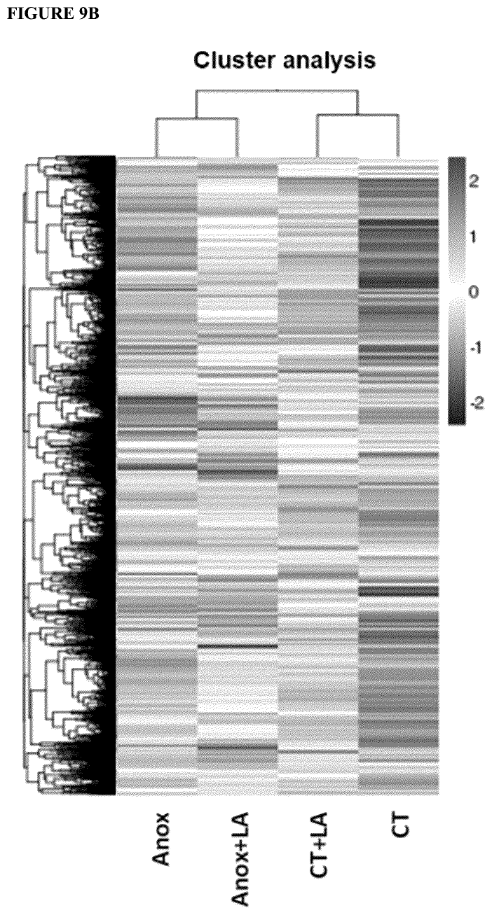

Different expression analyses revealed the presence of 12,999 clusters, out of which 11,925 were shared among control (CT), control with LA (CT+LA), anoxia (Anox), and anoxia with LA (Anox+LA). See FIG. 9B. These analyses also show the most significant differentially expressed genes (as depicted in Table 1) between CT vs. CT+LA and Anox vs. Anox+LA.

TABLE 1
Most significant DEGs in control vs. those exposed to LA for 3 h.

| | | Expression | |
|---|---|---|---|
| | | compared | |
| Gene name | Annotation | to control | padj |
| DDIT4 | DNA damage inducible transcript_4 | Up | 1.16E−45 |
| EGR1 | early growth response 1 | Up | 1.30E−38 |
| H1F0 | H1 histone family member 0 | Up | 1.42E−33 |
| CEBPB | CCAAT/enhancer binding protein (C/EBP) beta | Up | 4.22E−29 |
| ID2 | inhibitor of DNA binding 2 dominant | Up | 2.05E−28 |
| | negative helix-loop-helix protein | | |
| ITGA2 | integrin alpha 2 | Down | 1.86E−115 |
| SAMD5 | sterile alpha motif domain containing_5 | Down | 3.66E−63 |
| MAT2A | methionine adenosyltransferase II alpha | Down | 2.36E−41 |
| IGFBP4 | insulin-like growth factor binding protein 4 | Down | 2.56E−40 |
| SYTL2 | synaptotagmin-like 2 | Down | 5.24E−40 |

Further analyses of Gene ontology annotation revealed a number of critical signaling pathways that were differentially expressed in cells treated with LA, including biological processes involved in establishment of cell polarity, molecular functions that regulate junctional structures, and cellular components associated with epithelial repair (cell leading edge, ruffle, and apical junctional complex). See Table 2.

TABLE 2
Most significant DEGs in anoxic cells vs. those exposed to LA for 3 h.

| | | Expression | |
|---|---|---|---|
| | | compared | |
| Gene name | Annotation | to control | padj |
| TNS1 | tensin 1 | Up | 1.02E−255 |
| NDRG1 | N-myc downstream regulated 1 | Up | 7.38E−230 |
| PFKFB4 | 6-phosphofructo-2-kinase/fructose- | Up | 2.49E−180 |
| | 26-biphosphatase 4 | | |
| SLC2A3 | solute carrier family 2 (facilitated | Up | 4.83E−131 |
| | glucose transporter) member 3 | | |
| JPH2 | junctophilin 2 | Up | 6.06E−100 |
| EGR1 | early growth response 1 | Down | 1.78E−100 |
| HES1 | hairy and enhancer of split 1 (Drosophila) | Down | 6.63E−96 |
| HSPH1 | heat shock 105 kDa/110 kDa protein 1 | Down | 2.92E−72 |
| SAMD5 | Sterile alpha motif domain containing 5 | Down | 1.87E−69 |
| AMOTL2 | angiomotin like 2 | Down | 3.77E−62 |

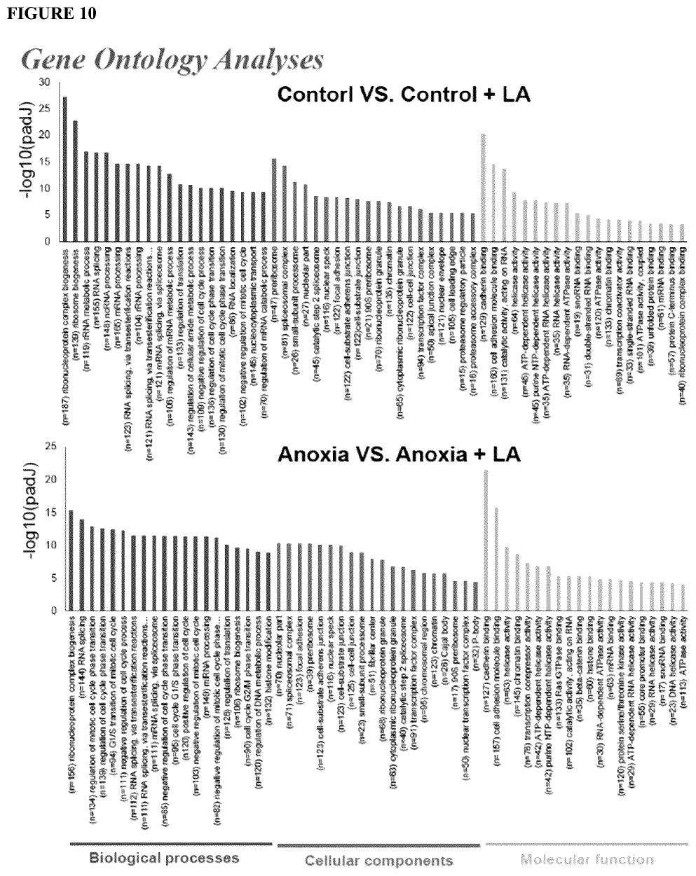

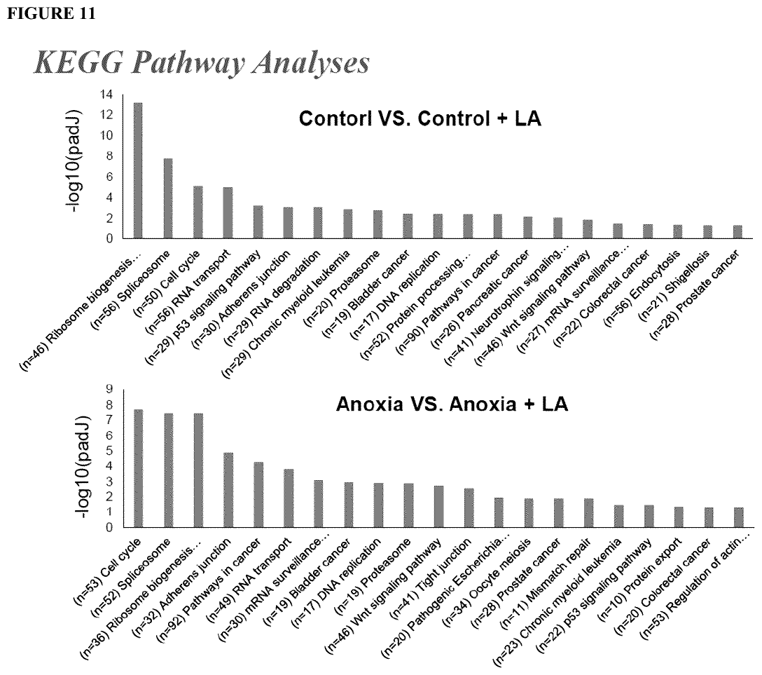

Furthermore, FIG. 10 shows that Ras/Rho GTPase-binding and protein serine/threonine kinase activity were differentially expressed in cells treated with LA. Additionally, Kyoto Encyclopedia of Genes and Genomes pathway enrichment analysis revealed enrichment of target genes for ‘cell cycle,’ ‘adherens junction’ and ‘Wnt signaling pathways. See FIG. 11.

Based on the GO analysis, various translational processes were significantly increased in LA-treated cells compared to non-treated cells. The analysis indicated that LA has a critical function in protein translational signaling. The treatment of LA also associated with various junctional pathways such as cadherin binding and cell adhesion. The KEEG pathways analysis showed that LA closely associated with cell cycling, adherens junctions, and translational pathways. The data including RNAseq analyses suggest that LA protects the tight junction barrier during A/R injury by regulating various cellular pathways including cell cycle, migration, and apical junctional complex.

### E. Evaluation of Barrier Function Under Normal Conditions

This experiment evaluated the role of LA in paracellular permeability during normal conditions.

For barrier function analysis, TEER was measured in monolayers of C2BBe1 cells treated with or without 10 mM LA. TEER was measured using a Chopstick Electrode Set (WPI, LLC, Sarasota, FL) on the basal and apical sides of a monolayer and attached to an Epithelial Volt Ohm Meter2 (WPI, LLC, Sarasota, FL).

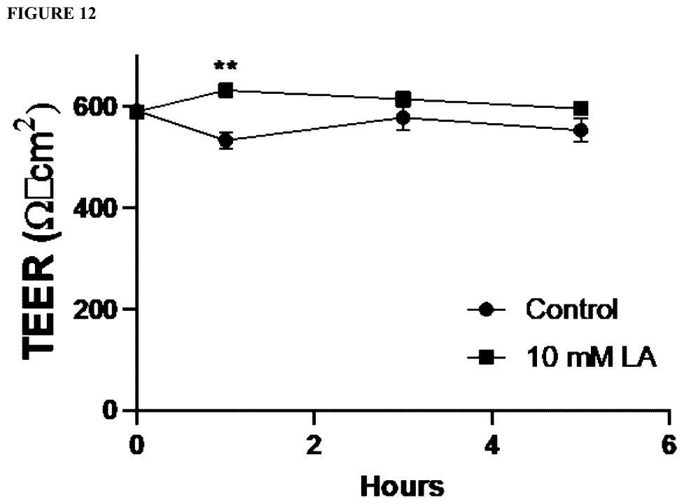

The results depicted in FIG. 12 show that treatment with 10 mM LA increased TEER in C2BBe1 monolayers, demonstrating that LA has a function in tightening paracellular pores under normal conditions as well as during A/R injury. LA has a function in tightening cell-to-cell junctional structures in normal conditions.

### F. Evaluation of Proliferation and Migration

This experiment evaluated the role of LA in proliferation and migration. Specifically, proliferation of non-treated or LA-treated (10 mM) C2BBe1 cells was evaluated by the CCK-8 assay kit (Dojindo Molecular Technologies, Rockville, MD) according to the manufacturer's instructions. 10 mM LA was treated every other day to cells seeded in 96-well culture plates. Viable cells were evaluated with the CCK-8 Assay Kit for five consecutive days. Migration was assessed by seeding C2BBe1 cells in 3 well culture-inserts (ibidi GmbH, Gräfelfing, Germany) and removing the inserts after reaching confluence to create a “wound” in the cell monolayer. 10 mM LA was added to serum-free and regular media to assess migration and proliferation, respectively. Images were captured at 0, 4, 8, 24, and 48 hours with Axio Vert A1 microscope (Carl Zeiss AG, Oberkochen, Germany) and closing of the wound was analyzed by measuring migration distance with ImageJ software.

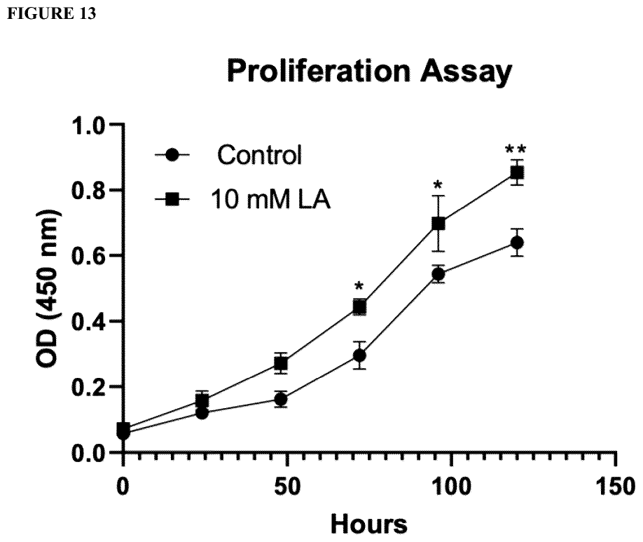

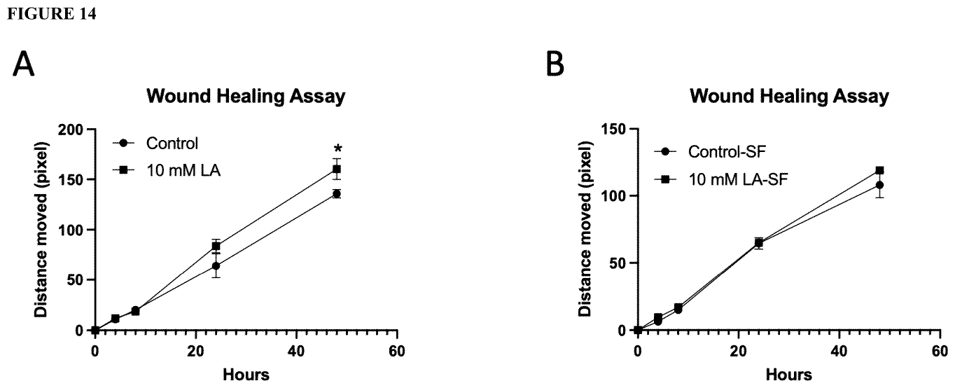

FIG. 13 shows that treatment with 10 mM LA in C2BBe significantly increased proliferation measured by CCK8. Proliferation of 10 mM LA-treated cells showed a significant increase comparted to non-treated C2BBe1 cells. Furthermore, FIG. 14 shows that the addition of LA did not significantly alter migration as evidenced by serum free media conditions.

The results suggest that LA facilitates cell proliferation but not cell migration. This proliferation induced by LA may promote the repair mechanism from intestinal injury.

What is claimed is:

1. A method for treating a subject for a condition characterized by Rho-associated coiled-coil kinase (ROCK) activity, comprising:
administering an effective amount of larazotide or larazotide derivative, or pharmaceutically acceptable salt thereof, to said subject in an amount and manner effective to inhibit ROCK activity in a tissue.

2. The method of claim 1, wherein the tissue is the gastrointestinal tract.

3. The method of claim 2, wherein the condition is selected from cancer, adenoma, celiac disease, inflammatory bowel disease (IBD), Crohn's Disease, Ulcerative Colitis, environmental enteropathy, esophagitis, necrotizing enterocolitis, intestinal ischemia, inflammatory liver disease, kidney disease, pancreatitis, hyperglycemia, and pulmonary or cardiac inflammation or fibrosis.

4. The method of claim 3, wherein the cancer originates from any tissue, optionally wherein the cancer originates from skin, colon, breast, lung, brain, bone, pancreas, kidney, liver, bladder, ovaries, testes, or prostate.

5. The method of claim 3, wherein the cancer is optionally colorectal cancer, leukemia, myeloma, or lymphoma.

6. The method of claim 1, wherein the subject is at risk for colorectal cancer.

7. The method of claim 6, wherein the subject's family history, genetic mutations, and/or health history increases the risk for colorectal cancer.

8. The method of claim 7, wherein a member of the subject's family has or had familial adenomatous polyposis (FAP), hereditary non-polyposis colorectal cancer (HNPCC), Peutz-Jeghers syndrome, or MUTYH-associated polyposis (MAP).

9. The method of claim 7, wherein the subject has a mutation of one or more genes selected from APC, MLH1, MSH2, MSH6, PMS2, EPCAM, STK11 (LKB1), and MUTYH that increases risk for colorectal cancer.

10. The method of any one of claims 6 to 9, wherein the subject has one or more single-nucleotide polymorphisms (SNPs) selected from rs6983267, rs4939827, rs3802842, rs16892766, rs10795668, rs4444235, rs10411210, rs6691170, rs4925386, rs3824999, rs647161, rs2423279, rs3217810, and rs59336.

11. The method of claim 1, wherein the tissue is cancer, which is optionally primary or metastatic cancer.

12. The method of claim 11, wherein the cancer is selected from lung cancer, breast cancer, kidney cancer, liver cancer, prostate cancer, cervical cancer, colorectal cancer, pancreatic cancer, melanoma, ovarian cancer, bone cancer, urothelial cancer, gastric cancer, head and neck cancer, glioblastoma, head and neck squamous cell carcinoma (HNSCC), non-small cell lung carcinoma (NSCLC), small cell lung cancer (SCLC), bladder cancer, hormone-refractory prostate cancer, and lymphoma.

13. The method of claim 11 or 12, wherein the tissue is metastatic cancer.

14. The method of claim 13, wherein the patient has metastatic melanoma.

15. The method of any one of claims 11 to 14, wherein the cancer is sarcoma or carcinoma.

16. The method of any one of claims 11 to 15, wherein the subject is undergoing or has undergone a cancer treatment selected from one or more of chemotherapy, radiation, resection, and immunotherapy and immune-oncology agents, optionally wherein the cancer immunotherapy is therapy with an immune checkpoint inhibitor.

17. The method of any one of claims 11 to 16, wherein the larazotide or larazotide derivative is formulated for intratumoral administration.

18. The method of claim 1, wherein the tissue is the eye.

19. The method of claim 18, wherein the larazotide or larazotide derivative is formulated for ophthalmic administration, optionally to the ocular surface or intraocular administration to the back of the eye.

20. The method of claim 1, wherein the larazotide or larazotide derivative is formulated for administration to the nasal and/or sinus cavity.

21. The method of claim 1, wherein the larazotide or larazotide derivative is formulated for administration to the ear canal.

22. The method of claim 1, wherein the tissue is the respiratory tract, and the subject optionally has a condition selected from asthma, chronic obstructive pulmonary disease (COPD), pulmonary fibrosis, cystic fibrosis, acute lung injury (ALI), acute respiratory distress syndrome (ARDS), emphysema, bronchitis, pneumonia, lung cancer, and respiratory infection.

23. The method of claim 22, wherein the larazotide or larazotide derivative is formulated for administration as a solution aerosol or powder to the lungs.

24. The method of clam 1, wherein the tissue is the vasculature, and the subject optionally has a condition selected from cardiac fibrosis, cardiac hypertrophy, hypertension, pulmonary hypertension, angina pectoris, vasospastic angina, heart failure, and stroke.

25. The method of claim 24, wherein the larazotide or larazotide derivative is formulated for pulmonary administration.

26. The method of any one of claims 1 to 25, wherein the subject has a ROCK mutation selected from one or more of Val1309, Tyr405, Ser1126, Pro1193S, relative to ROCK1 isoform, and/or Thr431Asn, Asp601 Val, and Lys1083Met, relative to ROCK2 isoform.

27. The method of any one of the preceding claims, wherein the subject is administered larazotide derivative, or a pharmaceutically acceptable salt thereof.

28. The method of claim 27, wherein the larazotide derivative comprises one or more modifications that increase ROCK inhibitor activity as compared to larazotide.

29. The method of claim 27, wherein the larazotide derivative comprises at least one, at least two, at least three, at least four, at least five D-amino acids.

30. The method of claim 29, wherein each amino acid of the larazotide derivative (other than Gly) is a D-amino acid, and the derivative is optionally a retro-inverso larazotide.

31. The method of any one of the preceding claims, wherein the larazotide or larazotide derivative is locally administered to the tissue.

32. A pharmaceutical composition comprising an effective amount of a peptide having the amino acid sequence Gly-Gly-Val-Leu-Val-Gln-Pro-Gly (SEQ ID NO: 1) for inhibiting ROCK activity in a tissue, or a pharmaceutically acceptable salt thereof, and a pharmaceutically-acceptable carrier.
